(19) United States
(12) Patent Application Publication
(10) Pub. No.: US 2011/0105593 A1
(43) Pub. Date: May 5, 2011

(54) MicroRNA-Formatted Multitarget Interfering RNA Vector Constructs and Methods of Using The Same

(75) Inventors: Laura Steel, Glenside, PA (US); Catherine J. Pachuk, Horsham, PA (US)

(73) Assignee: Drexel University

(21) Appl. No.: 12/793,248

(22) Filed: Jun. 3, 2010

Related U.S. Application Data
(63) Continuation of application No. 12/445,388, now abandoned.
(60) Provisional application No. 60/907,651, filed on Apr. 12, 2007.
(60) Provisional application No. 60/850,906, filed on Oct. 11, 2006.

(51) Int. Cl. A61K 31/7088; C12N 15/63; C12N 15/85; A61P 31/20; A61P 31/14

(52) U.S. Cl. 514/44 R; 435/320.1; 435/455

(57) ABSTRACT

Vectors expressing multiple microRNA (miRNA)-formatted interfering RNAs from a single transcript are disclosed and methods of using the same to inhibit expression of one or more target genes.

### CROSS-REFERENCE TO RELATED APPLICATIONS

This is continuation of U.S. patent application Ser. No. 12/445,388 filed Apr. 13, 2009 which is a National Stage application of PCT International Application No. PCT/US2007/081103, filed Oct. 11, 2007, which in turn claims the benefit pursuant to 35 U.S.C. §119(e) of U.S. Provisional Application No. 60/907,651, filed on Apr. 12, 2007 and U.S. Provisional Application No. 60/850,906, filed on Oct. 11, 2006, all of which are hereby incorporated by reference in their entirety herein.

### STATEMENT OF GOVERNMENT SUPPORT

This invention was made with government support from the National Institute of Health, grant number AI053988. The government has certain rights in the invention.

### FIELD OF THE INVENTION

The present invention relates to the field of gene expression, and more specifically to nucleic acid-based technologies for inhibiting expression of target genes. The invention encompasses vectors for mediating RNA interference via the microRNA pathway and methods for using the same to inhibit both endogenous and pathogen gene expression in mammals and mammalian cells.

### BACKGROUND OF THE INVENTION

RNA interference (RNAi) is an endogenous cell process that can suppress gene expression in a highly specific manner and it is providing new approaches for the development of therapies for viral infections and other diseases [20]. RNAi-based anti-viral strategies are particularly attractive since infection produces a unique set of viral transcripts that can serve as therapeutic targets. As an example, certain aspects of hepatitis B virus (HBV) biology and pathology make it a good candidate for RNAi-based therapies [1, 5]. The virus replicates through an RNA intermediate, so interfering RNAs can directly down-regulate viral replication and the production of infectious viral particles. In addition, a significant element in immune response-mediated HBV pathogenesis and persistence is the large excess of viral antigens, relative to infectious particles, produced in hepatocytes from either episomal or integrated forms of the viral genome [6]. The ability to reduce viral antigen production by silencing viral mRNAs is an important feature of RNAi-based strategies that is not shared by nucleoside analog inhibitors of the HBV polymerase [5].

The potential value of RNAi-based approaches in treating HBV infection has been demonstrated in many studies that have used interfering RNAs to down-regulate viral transcripts in both cell culture and animal models of infection (reviewed in [7]). Interfering RNAs that target HBV can be transfected into cultured hepatocytes either as pre-formed, synthetic short interfering RNAs (siRNAs) [8], or as plasmids that express short hairpin RNAs (shRNAs) [5, 9-12]. In a variety of mouse models of infection, HBV-targeted interfering RNAs have reduced viral antigen, transcript, or DNA production when delivered as siRNAs or as shRNAs expressed from plasmid or viral vectors [5, 13-17]. While many methods have been successful in laboratory studies, it remains a challenge to deliver RNAi agents to hepatocytes in a manner that will be clinically relevant in human patients.

Therapeutic interfering RNA can be directly introduced into cells as exogenously produced small interfering RNA (siRNA), or can be expressed within cells from vector-based systems, as eiRNA (expressed interfering RNA). Silencing mediated by synthetic siRNAs has been shown to be highly effective [21, 22], although it can be short-lived in in vivo applications [13]. Recent progress in chemical modification of siRNAs has improved both the longevity and specificity of these agents [23-25]. Nevertheless, plasmid or virus based vectors designed for cellular expression of interfering RNAs still provide the longest lasting effects and may therefore be most appropriate for the treatment of chronic diseases.

In early work on vector-based RNAi, pol III promoters were chosen to drive the expression of shRNAs that can be processed by intracellular enzymes into mature interfering RNAs [26-29]. The strong and ubiquitously active pol III promoters, with defined start and stop sequences, are well-suited for transcription of these short RNAs. Since then, however, as an understanding of the organization, expression, and processing of microRNAs (miRNAs) has grown (see FIG. 2), these endogenously encoded RNAs have provided another model for vector-based expression of interfering RNAs. Of particular importance was the finding that when an endogenous miRNA is redesigned so that it has full complementarity to a target of choice, it can mediate the degradation of the target RNA; and, silencing activity is retained if the stem-loop region, plus some flanking sequence, is placed into the context of an irrelevant RNA [30-32]. Further, it was found that miRNAs are often encoded in clusters where multiple miRNAs are processed from a single transcript [33-35] and that transcription is naturally driven by pol II promoters [36, 37].

In previous work, a therapeutic silencing vector for HBV with four different pol III driven sh-eiRNAs showed efficacy in cell culture and animals models of infection [5]. See PCT/US2005/029976, which is herein incorporated by reference in its entirety. In the second generation vector system described here, we have incorporated several aspects of miRNA expression to take advantage of its potential for polycistronic and tissue-specific expression. The miRNA formatted multitarget expression constructs described herein are ideal for treating viral infections characterized by a high rate of mutation, as multiple viral-targeted miRNAs within a single construct significantly decrease the chance of viral escape mutants and broaden the range of viral genetic variants that can be targeted.

In recent years, other groups have developed multitarget miRNA-based vector constructs for use in mammalian cells [42, 43, 48, 49]. However, no one to the present inventor's knowledge has yet to demonstrate inhibition of target gene expression in a mammal, and more specifically tissue-specific inhibition of viral gene expression in a mammal, using a miRNA-formatted multitarget expression construct. In fact, Zhou et al. recently reported that expressing two tandem copies of a miR-30-based synthetic miRNA in a single transcription unit was less effective for RNAi than a single copy of the same miRNA, suggesting that multitarget miRNA based constructs might not provide any benefit over single targeting constructs [50]. The vector constructs described in the present invention overcome the deficiencies of the prior art vectors, and are shown to be surprisingly effective in mammalian cells when expressed in a tissue-specific manner.

### SUMMARY OF THE INVENTION

The present invention provides nucleic acid vectors encoding multiple expressed microRNA (miRNA)-formatted interfering RNAs specific for one or more target genes, and methods of using the same to inhibit gene expression and treat a variety of diseases and infections in mammals. In one embodiment, among others, the invention provides a vector encoding at least two miRNA-formatted interfering RNAs specific for one or more target genes. In certain embodiments, the nucleic acid encoding the two or more miRNA-formatted interfering RNAs is operably linked to a single tissue-specific pol II promoter. In contrast to other miRNA-based constructs reported in the literature, the constructs of the present invention decrease or inhibit expression of the target gene(s) in a mammalian cell at least as effectively or more effectively than a vector expressing either of said miRNA-formatted interfering RNAs alone.

The vectors are conveniently designed so that single miRNA regions may be easily and sequentially inserted to construct vectors containing at least about two, three, four, five, six, seven, eight, nine, ten, etc. or more miRNA-formatted regions. The vector backbone, containing the miRNA 5′ and 3′ arms prior to the cloning of the stem-loop regions, is also included in the present invention. The vector backbone may be designed with restriction cloning sites between the 5′ and 3′ miRNA arm regions, particularly restriction cloning sites that enable successive cloning of individual miRNA units.

Also included in the invention are methods of inhibiting or decreasing expression of at least one target gene in a mammalian cell in vitro or in vivo comprising delivering to said cell a nucleic acid vector of the invention such that the nucleic acid encoding at least two microRNA (miRNA)-formatted interfering RNAs is expressed and expression of said at least one target gene is inhibited or decreased. The methods of the invention may be used to inhibit either endogenous mammalian genes, for instance genes involved in cancer or autoimmunity, or genes of mammalian pathogens including viruses.

The vectors described herein will help to maximize the efficacy of expressed interfering RNAs (eiRNAs) by increasing their potency and range of targets by allowing multiple eiRNAs to be produced from a single therapeutic vector. At the same time, the use of RNA pol II promoters can help in directing the therapeutic activity of these agents to specific cell types in an organism, even in the absence of precisely targeted delivery. Since the miR-eiRNA expression cassette described herein is built into non-protein coding RNA, co-expression of a protein product is not necessary, and this is a desirable feature for therapeutic vectors. While we have designed our vector to target HBV as an example, the general format of the disclosed miR-eiRNA vectors should be useable for silencing the expression of additional cellular or disease related genes.

### BRIEF DESCRIPTION OF THE DRAWINGS

FIG. 1. Four major transcripts from the HBV genome are 3′-coterminal and encode both the HBV pregenome and HBV proteins. (a) Schematic representation of overlapping open reading frames (ORFs) that encode the HBV proteins. (b) HBV transcripts are aligned with the ORF map, above. The protein products and size, in kilobases, of each transcript are indicated to the right. Vertical lines marked A-E show the location of five conserved regions that can be present in multiple transcripts.

FIG. 2 is a schematic diagram of an RNA interference pathway (figure from Cullen, B. R. (2006). Viruses and microRNAs. Nature Genetics Suppl 38: S25-S30).

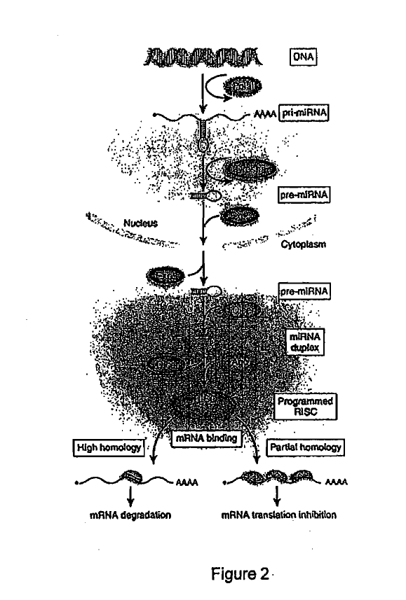

FIG. 3 is a depiction of multiple shRNA expression units encoded on a single vector. Each shRNA is expressed from its own RNA polymerase III promoter (7SK or U6) in this multigenic expression plasmid.

FIG. 4, comprising FIGS. 4A, 4B and 4C, is a series of blots demonstrating that viral RNAs, DNA replicative intermediates, and surface antigen proteins are coordinately reduced by expression of shRNAs. FIG. 4A is a Northern blot analysis of RNA isolated from HepG2 cells transfected with pHBV2 (a plasmid that expresses HBV) plus increasing amounts of a tri-genic shRNA expression plasmid. Viral transcripts, as indicated to the right, were detected by probing with a radio labeled HBV fragment. The lower panel shows the ethidium bromide stained gel as a loading control. FIG. 4B is a Southern blot analysis of HBV DNA replicative intermediates. DNA was isolated from transfected cells and probed as in FIG. 4A; rcDNA is relaxed circular DNA; dlDNA is double-stranded linear DNA; and ssDNA is single-stranded DNA. FIG. 4C is an Immunoblot analysis of proteins from cells transfected as in FIG. 4A. The large (L) and middle (M) surface antigens were detected using antibody to the preS2 region of HBsAg.

FIG. 5 is a chart demonstrating the reduction of serum HBsAg levels after intravenous delivery of dCS (cholesterol spermine)-formulated shRNA expression plasmid in a mouse model of HBV infection.

FIG. 6 is a schematic diagram depicting the design for a plasmid that encodes multiple, different miRNAs. Following transcription, the stem-loop regions are processed into individual miRNAs.

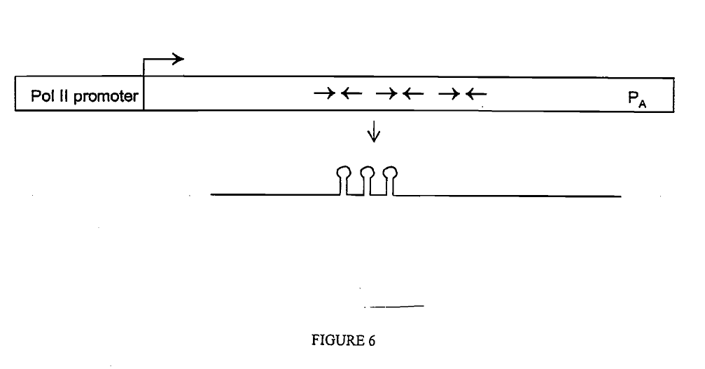

FIG. 7. Silencing activity of plasmids that express miR-formatted interfering RNAs that target HBV RNAs. (a) General structure of the U6 RNA pol III expression cassette for miR-30 formatted interfering RNAs in pUC-U6-30/XX plasmids. Approximately 125 nt of sequence on each side of the stem structure are derived from miR-30 (open boxes). Also, 19 nt of loop sequence (open box between shaded boxes) was derived in part from miR-30 (the first two bases (CT) and last two bases (GG) of the natural loop region have each been changed to CT). The stem region (shaded boxes with opposing arrows) may be designed with significant or complete complementarity to sequence in the target RNA. The U6 promoter (stippled box) and pol III transcription termination site (T₆) are indicated. (b) Silencing activity from pUC-U6-30/1737 and pUC-U6-30/EGFP. Huh7 cells were transfected with constant amounts of pHBV/2 (84 pM, equal to 500 ng in 1 ml) and pM1-SEAP together with increasing amounts of the indicated silencing plasmid. The amounts are shown as pM and correspond to 10, 50, and 100 ng used in a 1 ml transfection. Culture supernatant was collected 48 hr post-transfection and assayed for SEAP activity and HBsAg. Results are calculated as HBsAg in the culture supernatant, normalized to SEAP activity and expressed as a percent of control wells where no silencing plasmid was added. (c) Silencing activity from pUC-U6-30/XX plasmids that target slightly shifted regions near the original sites at 1737 and 1907. Huh7 cells were transfected as described for panel b, with increasing amounts of silencing plasmid. Amounts are indicated as pM and correspond to 10, 50, and 200 ng of silencing plasmid in a 1 ml transfection. For both (b) and (c), HBsAg values are the average of two assays each for two independent transfections.

FIG. 8. Silencing activity of miR-eiRNAs expressed from RNA pol II promoters. (a) General structure of the pol II expression cassette. A pol II promoter (either the CMV-IE promoter or the liver specific promoter of pLIVE) drives the expression of a transcript that contains two introns (open boxes) and no ORF. A stem-loop region with ˜30 bp of flanking sequence, copied from pUC-U6-30/XX plasmids, is inserted into the second intron (shaded boxes with opposing arrows). One (top) or more (bottom) stem-loops can be inserted into the intron. The start site of transcription (bent arrow) and the polyadenylation site (vertical arrow) are indicated. (b) Silencing activity from RNA pol II driven miR-eiRNAs. Huh7 cells were transfected with a constant amount of psiCH2-HBV21/20 (101 pM, 250 ng in 0.5 ml) together with increasing doses of the indicated silencing plasmid. Amounts are shown as pM and correspond to 25, 50, and 100 ng of silencing plasmid in a 0.5 ml transfection. Two days post-transfection, cells were lysed and assayed for Renilla and firefly luciferase activities. Results are expressed as the ratio of Renilla to firefly luciferase activity, normalized to results from cells with no added silencing plasmid (‘none’). Each value represents the average of two assays each for two independent transfections.

FIG. 9. Both eiRNAs expressed from a bicistronic plasmid are active. Silencing activity of pLV-30s/1737B/1907A was tested against two different reporter plasmids. In panel a, the dual luciferase reporter plasmid psiCH-HBV23/20 contains HBV target sequence from the 1737 region. In panel b, the reporter psiCH-HBV22/24 contains HBV target sequence from the 1907 region. Transfections were as described in FIG. 8, with 118 pM (250 ng in 0.5 ml) target plasmid and 0, 10, 25, 50, 100, or 200 ng silencing plasmid. Each value represents the average of three assays for two independent transfections, +/−SD.

FIG. 10. Mature interfering RNAs are efficiently processed from both cistrons of a bicistronic expression plasmid. Panels (a-c): RNA was isolated from Huh7 cells transfected with miR-eiRNA plasmids and analyzed by northern blotting. Cells were transfected as follows: lane 1) pLV-30s/1737B; lane 2) pLV-30s/1907A; lane 3) pLV-30s/1737B/1907A; lane 4) pLV-30s/EGFP. Lane M contains radiolabeled RNA markers, with sizes indicated in nt. Radiolabeled oligonucleotides were used to probe the same blot, sequentially, for (a) U6 RNA, gel loading control, (b) the anti-sense strand of 1737B, and (c) the anti-sense strand of 1907A. RNA from cells transfected with pLV-30s/EGFP serves as a control for hybridization specificity. In panel (d), small RNAs were isolated from Huh7 cells transfected with pUC-U6-30/1737A (lane 1), p7SK-sh 1737 (lane 2), pUC-U6-30/EGFP (lane 3), or untransfected cells (lane 4). The blot was probed with a radiolabeled oligonucleotide detecting the anti-sense (guide) strand of 1737.

FIG. 11. Tissue specificity of silencing is determined by the pol II promoter. HeLa cells were transfected with the psiCH2-HBV21/20 reporter plasmid (58 pM) together with increasing amounts of pLV-30s/1737B/1907A or pLV/CMV-30s/1737B/1907A (from 4 to 40 pM), as indicated. Two days post-transfection, cells were assayed for Renilla and firefly luciferase activity, as in FIG. 8. Each value represents the average of two assays for four independent transfections, +/−SD.

FIG. 12. Expression of miR-eiRNAs does not induce an interferon response. Total RNA was isolated from HeLa cells 6 hr and 24 hr after transfection with 2 μg/ml poly(I:C), mock transfection, transfection with pCMV-LV (no miR-eiRNA), or transfection with pCMV-30s/1737B/1907A. Silencing plasmids were used at 51 pM. Quantitative RT-PCR was used to measure levels of (a) p56 mRNA, (b) IFN-β mRNA, and (c) MX-1 mRNA. Results are presented as expression relative to levels found in untreated HeLa cells and represent the average of three reactions.

FIG. 13. The indicated silencing plasmids were coinjected with a reporter target plasmid (based on psiCHECK2) that contains all four of the regions targeted by Nuc050, the multi-genic sh-eiRNA silencing plasmid shown in FIG. 3. The reporter target plasmid together with 0.1 μg of silencing plasmid was introduced into NOD-SCID mice by hydrodynamic injection into the tail vein. Groups of 10 mice, or 8 mice for LS-005, were injected for each plasmid and livers were collected 5 days post-injection for assay of Renilla and firefly luciferase. Silencing activity was measured as a reduction in Renilla luciferase activity relative to firefly luciferase activity. Results are presented as both the percent knockdown (A) and mean renilla; firefly RLU ratios (B) observed.

FIG. 14. Plasmids that express four different eiRNAs show more potent silencing than plasmids that express two eiRNAs. a) A schematic depiction of expression units encoding either two or four miRNA-formatted interfering RNAs. The plasmid pLV-30s/1737B/1907A is in the form shown at the top and is called here pLVD. In the plasmid pLVQ, in the form shown at the bottom, two additional eiRNA stem-loops have been added so that four different regions in HBV RNAs are now targeted. b) Silencing activity from pLVD and pLVQ. Huh7 cells were co-transfected with a constant amount of the dual luciferase reporter, psiCH-HBV, and increasing amounts of pLVD or pLVQ. Results are expressed as the ratio of Renilla to firefly luciferase activity, normalized to results from cells with no added silencing plasmid (“control”).

FIG. 15. Sequences encoding miR-formatted interfering RNAs can be placed in intronic or exonic regions of a transcript that does not co-express a protein product. Panels a, b, and c depict expression units that encode multiple eiRNA stem-loops, shown as shaded boxes with opposing arrows, that are placed within the second intron (panel a), the first intron (panel b), or the exon region (panel c). Panel (d) shows the silencing activity of plasmids carrying each of these expression units. Huh7 cells were co-transfected with a constant amount of the dual luciferase reporter, psiCH-HBV, and increasing amounts of each of the indicated silencing plasmids. Results are expressed as the ratio of Renilla to firefly luciferase activity, normalized to results from cells with no added silencing plasmid (“control”).

FIG. 16. There is no loss of functionality from individual eiRNAs when expressed from a multi-cistronic plasmid as compared to expression from a corresponding mono-cistronic plasmid. Section A: Huh7 cells were co-transfected with a dual luciferase psiCH-HBV reporter plasmid carrying target sequence for HBV region 1737, 1907, 799, or 2791 (as indicated along the X-axis) together with the corresponding mono-cistronic silencing plasmid targeting each region. Section B: Huh7 cells were co-transfected with the indicated psiCH-HBV reporter target plasmid together with the multi-cistronic silencing plasmid pLVQ-Int2. Section C: Huh7 cells were co-transfected with the indicated psiCH-HBV reporter target plasmid together with the multi-cistronic silencing plasmid pLVQ-Ex.

### DETAILED DESCRIPTION OF THE INVENTION

The present invention provides nucleic acid vectors encoding multiple expressed miRNA-formatted interfering RNAs specific for one or more target genes. The phrase “miRNA-formatted interfering RNA” refers to an RNA having a secondary structure comprising a double stranded, target-specific stem-loop region flanked by miRNA arm regions, wherein the miRNA arm regions have sequences taken from a naturally occurring miRNA, or sequences that are predicted to form a secondary structure that models the structure of a naturally occurring miRNA. Depending on the naturally occurring miRNA, the first 9 to 13 bases of the arm regions typically have some potential for secondary structure, and the rest of the arm sequence is relatively unstructured. A consensus sequence or format for the microRNA arm regions has been reported. See Han, J., et al. (2006), Molecular basis for the recognition of primary microRNAs by the Drosha-DGCR8 complex. Cell 125: 887-901, which is herein incorporated by reference in its entirety.

The double stranded stem region is designed to be specific for the target gene. This means that the double stranded stem region of the expressed miRNA contains one strand that is at least substantially complementary to a sequence in the target gene, and the other strand is at least substantially identical to the complementary sequence in the target gene, substituting uracil for any thymidine residues. “Substantially” means at least about 75%, 80%, 85%, 90%, 95%, 96%, 97%, 98%, or 99%, or sufficiently complementary or identical to the target gene to modulate expression of the target gene in a target-specific manner. In some embodiments, the double stranded stem region is completely complementary and identical to a sequence of the target gene. In some embodiments, expression of the miRNA-formatted interfering RNAs results in degradation of the target mRNA via the RNA interference pathway. In other embodiments, expression of the miRNA-formatted interfering RNAs may lead to translational arrest of the target mRNA or a mixture of translational arrest and degradation.

In one embodiment, the double stranded stem region is about 22 base pairs, but may range in length from about 19 to about 29, about 21 to about 27 or about 20 to about 25 base pairs, i.e., 19, 20, 21, 22, 23, 25, 25, 26, 27, 28, 29, or longer. The two portions of the stem region can be completely complementary. The two portions of the stem region may also be partially complementary, i.e., at least about 50%, 60%, 70%, 80%, 90%, 95% or 99% complementary. When completely complementary, the two portions of the stem are encoded by a DNA construct containing an inverted repeat of the sequences in the stem region, separated by the “loop” region, such that transcription of the DNA construct produces a RNA that forms a stem-loop structure.

The loop region is approximately 19 ribonucleotides, but may range in length from about 4 to about 30 ribonucleotides. The loop region is between and connects the two arms of the stem region such that complementary base pairing of the stem region forces the intervening region to “loop” out. In contrast to the miRNA arms, the loop region between the two portions of the stem has minimal potential for base pairing.

In some embodiments, among others, the miRNA arm regions are about 20 to 45 nucleotides. In other embodiments, the miRNA arms are about 25 to about 40 nucleotides. The length of the miRNA arms may vary depending on the microRNA species used to make or model the constructs. For instance, the present inventor has determined that for constructs encoding multiple miR-30-formatted interfering RNAs, about 36 nucleotides is sufficient for the 5′ miRNA arm, and about 28 nucleotides for the 3′ miRNA arm. However, depending on the miRNA, other lengths for the 5′ and 3′ arm regions could also be suitable, including 5′ and/or 3′ arm regions of about 25, 26, 27, 28, 29, 30, 31, 32, 33, 34, 35, 36, 37, 38, 39 or 40 nucleotides.

MiR-30 is a naturally occurring miRNA encoded in the third intron of an mRNA-like non-coding RNA found on human chromosome 6 [53]. This region naturally encodes a single miRNA, not a cluster of multiple miRNAs, although the present inventor has demonstrated that miR-30 sequences can form the basis of multi-miRNA expression constructs. However, other miRNAs may be used in the constructs of the present invention, including but not limited to miRNAs naturally encoded either singly or in clusters, those encoded in exons of mRNAs, exons of non-coding RNAs, introns of mRNAs, or introns of non-coding RNAs. Particular miRNAs that could be used in the methods of the invention include, but are not limited to, miR-1 through miR-34; miR-2-1; miR-92 through miR-101; miR-103 through miR-107; miR-109 through miR-114; miR-116; miR-119; miR-122; miR-125; miR-126; miR-127; miR-129; miR-130; miR-132; miR-133; miR-134; miR-136; miR-138; miR-140; miR-141; miR-144 through miR-151; miR-153; miR-154; miR-157; miR-158; miR-160; miR-162; miR-164; miR-172 through miR-180; miR-182 through miR-189; miR-191; miR-192; miR-193; miR-195; miR-196; miR-197; miR-199; miR-201; miR-203; miR-205; miR-224. Any suitable miRNA sequence may be used in the methods of the invention, such as any of those listed in the miRBase database, which is a database housed at the Wellcome Trust Sanger Institute that lists all miRNA sequences (and other associated information). See http:/microrna.sanger.ac.uk.

As noted above, the present invention includes vectors providing multiple miRNA-formatted interfering RNAs specific for one or more target genes. A “vector” is a composition of matter which can be used to deliver a nucleic acid of interest to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. Examples of viral vectors include, but are not limited to, adenoviral vectors, adeno-associated virus vectors, retroviral vectors, and the like. An expression construct can be replicated in a living cell, or it can be made synthetically. For purposes of this application, the terms “expression construct,” “expression vector,” and “vector,” are used interchangeably to demonstrate the application of the invention in a general, illustrative sense, and are not intended to limit the invention.

In one embodiment, among others, the invention provides a nucleic acid vector comprising a nucleic acid encoding at least two microRNA (miRNA)-formatted interfering RNAs specific for at least one target gene, wherein said nucleic acid encoding said at least two (miRNA)-formatted interfering RNAs is operably linked to a single pol II promoter. The phrase “under transcriptional control” or “operably linked” as used herein means that the promoter is in the correct location and orientation in relation to a polynucleotide to control the initiation of transcription by RNA polymerase and expression of the polynucleotide. Preferably, the nucleic acid vectors of the invention decrease expression of said at least one target gene in a mammalian cell at least as effectively or more effectively than a vector expressing either of said microRNA (miRNA)-formatted interfering RNAs alone. Such expression levels may be measured and/or quantified using any suitable method, for instance by detecting degradation of the target mRNA transcript, by quantifying or measuring a decrease in functional activity of the protein encoded by the target gene, or, in methods of treating infection, by measuring a decrease in the amount of target pathogen detectable in the mammal.

In addition to the attributes described above for the encoded miRNA-formatted interfering RNAs, the nucleic acid regions encoding each miRNA-formatted interfering RNA may be operably linked with a nucleic acid linker region. The intervening linker region may range from about 1 to about 5 to about 15 to about 20 nucleotides in length or longer. In some embodiments, the linker regions are not more than about 6 to 10 nucleotides. The linker regions may contain one or more restriction cloning sites for sequential cloning of multiple miRNA unit constructs. Any suitable restriction cloning site may be used. Particularly preferred are cloning sites that enable sequential cloning by creating overhangs that are compatible with other restriction enzymes, such as the XbaI/SpeI sites used in the Examples of the present invention. Other suitable, compatible restriction enzyme pairs are known in the art, i.e. SalI/XhoI; NdeI/AseI; BclI/BamHI/BglII/MboI; and AvaI/XmaI, to name a few. See also, for example, the chain reaction cloning methods taught in U.S. Pat. No. 6,143,527, incorporated herein by reference.

In some embodiments of the invention, the nucleic acid encoding said at least two (miRNA)-formatted interfering RNAs is not operably linked to a separate protein coding sequence. It is particularly surprising that the pol II expression constructs of the present invention are so effective in the absence of a linked, co-expressed protein coding sequence, since many researchers have reported that pol II expression constructs are not as effective in the absence of a separate protein coding region encoded on the same transcript as the miRNA [41].

In some embodiments, among others, the nucleic acid encoding said at least two miRNA-formatted interfering RNAs is located in a nucleic acid encoding an intron or in a nucleic acid encoding an untranslated region of an mRNA or in a non-coding RNA. In one embodiment, the nucleic acid encoding said at least two miRNA-formatted interfering RNAs is located in a nucleic acid encoding a functional intron, wherein the expression construct contains at least one other intron 5′ and/or 3′ to the intron containing the at least two miRNA-formatted interfering RNAs. In other embodiments, the expression construct encodes a single transcript with multiple functional intron regions, each encompassing one or more miRNA-formatted interfering RNAs. By “functional intron” is meant a sequence flanked by splice junctions or other sequences that facilitate functional splicing. In some embodiments, a nucleic acid encoding one or more miRNA-formatted interfering RNAs is located in an exon sequence. In other embodiments, said exon sequence containing one or more miRNA-formatted interfering RNAs is located between nucleic acid sequences encoding functional introns. The expression construct may encode a single transcript containing multiple intron regions separated by one or more exon regions, wherein miRNA-formatted interfering RNAs may be located in one or more of the intron and/or exon regions.

As described above, the miRNA-formatted interfering RNAs encoded by the expression vectors of the invention are specific for at least one target gene. In one embodiment, the target gene is an endogenous gene of a mammalian cell. In another embodiment, the target gene is a gene of a pathogen that infects a mammalian cell, including viruses and intracellular parasites. The multiple (two or more) miRNA-formatted interfering RNAs on a single expression construct may be the same miRNA repeated in succession. Alternatively, the multiple miRNAs may be different miRNAs that are specific for the same target gene. Alternatively, the multiple miRNAs may be different miRNAs that are specific for two or more target genes of the same cell or pathogen. Alternatively, the multiple miRNAs may be different miRNAs that are specific for target genes of different pathogens or pathogen variants, for instance target genes of different viruses or viral variants.

Viruses that may be targeted by the vectors of the present invention include but are not limited to Retroviridae (e.g. human immunodeficiency viruses, such as HIV-1 (also referred to as HTLV-III, LAV or HTLV-III/LAV, or HIV-III; and other isolates, such as HIV-LP)); Picornaviridae (e.g. polio viruses, hepatitis A virus; enteroviruses, human coxsackie viruses, rhinoviruses, echoviruses); Calciviridae (e.g. strains that cause gastroenteritis); Togaviridae (e.g. equine encephalitis viruses, rubella viruses); Flaviviridae (e.g. dengue viruses, encephalitis viruses, yellow fever viruses); Coronaviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviruses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus); Poxviridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

The miRNA formatted multitarget expression constructs described herein are particularly useful for treating viral infections characterized by a high rate of mutation, such as HIV and influenza, since multiple viral-targeted miRNAs within a single construct significantly decrease the chance of viral escape mutants and broaden the range of genetic viral variants that can be treated. Ideal target sequences that are conserved across different influenza viruses, including human, bird and swine viruses, have been identified, and may be used in the miRNA constructs of the present invention. See U.S. Provisional Application No. 60/907,650 entitled “Influenza Sirna Molecules, Expression Constructs, Compositions, And Methods Of Use”, filed Apr. 12, 2007, which is herein incorporated by reference in its entirety.

In one embodiment, the vectors of the present invention are designed to express one, two, three, four, five, or more HBV-specific miRNA-formatted interfering RNAs, for instance in a liver cell or in the liver of a mammal. Specific sequences in HBV that may be targeted are disclosed in the examples reported herein. Other target sequences are also known, and are disclosed in PCT/US2004/019229 and PCT/US2005/0046162, each of which is herein incorporated by reference in its entirety.

Endogenous genes that may be targeted by the methods of the present invention include genes involved in autoimmunity and/or cancer. The miRNA formatted multitarget expression constructs described herein are ideal for treating such diseases, which are typically characterized by the involvement of multiple genes.

The term “autoimmune disease” as used herein is defined as a disorder that results from an autoimmune response. An autoimmune disease is the result of an inappropriate and excessive response to a self-antigen. Examples of autoimmune diseases, include but are not limited to, Addision's disease, alopecia greata, ankylosing spondylitis, autoimmune hepatitis, autoimmune parotitis, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

In one embodiment of the invention, the multiple miRNA-formatted interfering RNAs are expressed on a single transcript from a pol II promoter. However, in other embodiments, promoters other than pol II might be used so long as the promoter facilitates expression of the multiple miRNA-formatted interfering RNAs and inhibition of target gene expression at least as effectively or more effectively than a vector expressing either of said microRNA (miRNA)-formatted interfering RNAs alone. Where a pol II promoter is used, the expression construct typically terminates with a polyadenylation signal. Examples of additional promoters are RNA polymerase I and III promoters.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product.

The promoters employed in the vector constructs of the invention may be constitutive or inducible. A “constitutive” promoter is a nucleotide sequence which, when operably linked with a polynucleotide which encodes or specifies a gene product, causes the gene product to be produced in a cell under most or all physiological conditions of the cell. An “inducible” or “regulatable” promoter is a nucleotide sequence which, when operably linked with a polynucleotide which encodes or specifies a gene product, causes the gene product to be produced in a cell substantially only when an inducer which corresponds to the promoter is present in the cell.

The promoters employed in the vector constructs of the invention may be tissue-specific or ubiquitous. A ubiquitous promoter is expressed in a broad range of cell types, and may include certain viral promoters, for instance, the CMV promoter. A “tissue-specific” promoter is one that is not universally expressed in all cells, and that substantially enhances expression of a gene in one or more particular tissues as compared with other tissues. The choice of tissue-specific pol II promoter will depend on the cell or tissue targeted for expression, or where a pathogen is targeted, on the cell or tissue infected by the pathogen. Suitable tissue-specific promoters are known in the art, and include but are not limited to the alpha fetoprotein enhancer/albumin promoter for liver-specific expression, the secretoglobin family 1A, member 1 promoter for expression in the bronchial epithelium, surfactant, pulmonary-associated proteins A, B, or C promoters for lung parenchymal expression, to name just a few. Liver-specific promoters are particularly useful for the HBV targeting constructs described herein. Such liver-specific promoters include but are not limited to the human alpha-1 antitrypsin (hAAT) promoter, the lecithin-cholesterol acyl transferase (LCAT) promoter, the apolipoprotein H (ApoH) promoter, and the prealbumin promoter, to name a few, alone or in combination with liver-specific enhancer regions such as that of the alpha fetoprotein or the enhancer of the gene for ApoE.

The expression vectors of the present invention include the backbone vectors for building the constructs described herein. For example, the present invention includes a nucleic acid vector for expressing one or more miRNA-formatted interfering RNAs comprising a tissue-specific pol II promoter operably linked to at least first and second intron sequences and a polyadenylation signal, wherein said second intron comprises 5′ and 3′ miRNA-formatted arm regions of no more than about 20 to about 45 consecutive nucleotides each flanking one or more cloning sites for recombinantly inserting a nucleic acid encoding a target-specific stem-loop construct having the attributes described above. The 5′ and 3′ arm regions are derived from a naturally occurring miRNA, or have a secondary structure modeled after a naturally occurring miRNA, as described above. In some embodiments, the vector further comprises one or more cloning restriction sites 5′ and/or 3′ of said miRNA-formatted arm regions for sequential cloning of multiple miRNA units as also described above.

The vector constructs of the present invention are useful in methods of inhibiting or decreasing expression of at least one target gene in a cell in vitro or in vivo. In the context of the present invention, “in vitro” means that an expression vector of the invention is expressed in a cell in culture. “In vivo” means that an expression vector of the invention is expressed in a cell in a mammal or other multicellular animal. The vector constructs of the invention may also be expressed in a test tube in the presence of cell extracts containing the necessary transcriptional machinery or proteins to generate the primary miRNA transcript (pri-miRNA), and contacted with the isolated Microprocessor complex containing the double-stranded RNA binding protein Pasha (also called DGCR8) and the RNase III enzyme Drosha in order to process the pri-miRNA transcript into pre-miRNAs, and RISC (RNA induced silencing complex) components for further processing into individual miRNAs [51,52]. The individual miRNAs may then be delivered to the target cell or tissue.

In one embodiment, the present invention includes a method comprising transfecting a cell containing the target gene or delivering to the cell the expression vector of the invention such that the nucleic acid encoding the two or more miRNA-formatted interfering RNAs is expressed in the cell and expression of said at least one target gene is inhibited or decreased. Preferably, expression of the target gene(s) is decreased at least as effectively or more effectively than with a vector expressing any of said microRNA (miRNA)-formatted interfering RNAs alone. The methods of the present invention are useful in methods of treating a patient infected with a target pathogen, or a patient in which target endogenous genes are aberrantly expressed. In one embodiment, the methods of the present invention include delivering to the patient a nucleic acid vector according to the present invention such that at least two miRNA-formatted interfering RNAs are expressed and expression of at least one target gene is inhibited or decreased.

Depending on the cell or tissue targeted by the methods of the invention, there will be multiple means of delivering the expression vector of the invention to the target tissue. For example, the vector can be readily introduced into a host cell, e.g., mammalian, bacterial, yeast or insect cell using any method in the art. For example, the expression vector can be transferred into a host cell by physical, chemical or biological means. Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art. Exemplary formulations are also disclosed in U.S. Pat. No. 5,981,505; U.S. Pat. Nos. 6,217,900; 6,383,512; U.S. Pat. No. 5,783,565; the Boutin patent family, U.S. Pat. No. 7,202,227; U.S. Pat. No. 6,379,965; U.S. Pat. No. 6,127,170; and U.S. Pat. No. 5,837,533; and WO03/093449, which are herein incorporated by reference in their entireties.

The invention also encompasses the use of pharmaceutical compositions of the appropriate vector to practice the methods of the invention. The pharmaceutical compositions useful for practicing the invention may be administered to deliver a dose of between 1 ng/kg/day and 100 mg/kg/day.

As used herein, the term “physiologically acceptable” ester or salt means an ester or salt form of the active ingredient which is compatible with any other ingredients of the pharmaceutical composition, which is not deleterious to the subject to which the composition is to be administered. The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit. Suitable carriers include, but are not limited to, saline, buffered saline, dextrose, water, glycerol, ethanol, and combinations thereof. The composition can be adapted for the mode of administration and can be in the form of, for example, a pill, tablet, capsule, spray, powder, or liquid.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, intravenous (IV), intra-arterial, intradermal, intrathecal, intramuscular (IM) and kidney dialytic infusion techniques. The compositions of the invention may also be administered, without limitation, topically, orally, and by mucosal routes of delivery such as intranasal, inhalation, rectal, vaginal, buccal, and sublingual.

The present invention may, in certain embodiments, employ the methods disclosed in the U.S. Provisional Application No. 60/907,014, filed Mar. 16, 2007 entitled “Methods and Compositions For Directing RNAi-Mediated Gene Silencing In Distal Organs Upon Intramuscular Administration Of DNA Expression Vectors,” which is hereby incorporated by reference in its entirety. Specifically, intramuscular injection or electroporation of expression constructs encoding dsRNA(s) results in targeted inhibition of gene expression in other organs and tissues of the body. Without being bound by any theory, the inventors thereof hypothesize that delivery of dsRNA to distal tissues such as respiratory epithelial cells, for example, may be mediated by extracellular vesicles (exovesicles) containing expressed dsRNA or injected siRNA or shRNA that bud from the surface of transfected muscle cells. In particular, it has been shown that dsRNA expressed in muscle cells is delivered in vivo to the liver. Targeting to other cells and tissues may be accomplished by coexpressing cell surface ligands that are incorporated into exovesicles and target such dsRNA containing exovesicles to the target cell or tissue. Such cell surface ligands may be coexpressed from the same vector as the multiple miRNA constructs, or from a separate expression vector. In this embodiment, the target gene is in a different cell than the transfected cell that expresses the vector encoding the multiple miRNAs.

It is known that some dsRNA sequences, possibly in certain cell types and through certain delivery methods, may result in an interferon response. The methods of the invention may be performed so as not to trigger an interferon/PKR response, by expressing the multiple miRNA-formatted interfering RNA molecules intracellularly from an expression vector. See US Published Application 20040152117, which is herein incorporated by reference. However, in embodiments where the miRNA constructs are not expressed intracellularly, the interferon/PKR response may also be inhibited by other means. For instance, interferon and PKR responses may be silenced in the transfected and target cells using a dsRNA species directed against the mRNAs that encode proteins involved in the response. Alternatively, interferon response promoters are silenced using dsRNA, or the expression of proteins or transcription factors that bind interferon response element (IRE) sequences is abolished using dsRNA or other known techniques.

By “under conditions that inhibit or prevent an interferon response or a dsRNA stress response” is meant conditions that prevent or inhibit one or more interferon responses or cellular RNA stress responses involving cell toxicity, cell death, an anti-proliferative response, or a decreased ability of a dsRNA to carry out a PTGS event. These responses include, but are not limited to, interferon induction (both Type 1 and Type II), induction of one or more interferon stimulated genes, PKR activation, 2′5′-OAS activation, and any downstream cellular and/or organismal sequelae that result from the activation/induction of one or more of these responses. By “organismal sequelae” is meant any effect(s) in a whole animal, organ, or more locally (e.g., at a site of injection) caused by the stress response. Exemplary manifestations include elevated cytokine production, local inflammation, and necrosis. Desirably the conditions that inhibit these responses are such that not more than 95%, 90%, 80%, 75%, 60%, 40%, or 25%, and most desirably not more than 10% of the cells undergo cell toxicity, cell death, or a decreased ability to carry out a PTGS event, compared to a cell not exposed to such interferon response inhibiting conditions, all other conditions being equal (e.g., same cell type, same transformation with the same dsRNA).

Apoptosis, interferon induction, 2′5′ OAS activation/induction, PKR induction/activation, anti-proliferative responses, and cytopathic effects are all indicators for the RNA stress response pathway. Exemplary assays that can be used to measure the induction of an RNA stress response as described herein include a TUNEL assay to detect apoptotic cells, ELISA assays to detect the induction of alpha, beta and gamma interferon, ribosomal RNA fragmentation analysis to detect activation of 2′5′ OAS, measurement of phosphorylated eIF2a as an indicator of PKR (protein kinase RNA inducible) activation, proliferation assays to detect changes in cellular proliferation, and microscopic analysis of cells to identify cellular cytopathic effects. See, e.g., US Published Application 20040152117, which is herein incorporated by reference.

The following examples are provided to describe and illustrate the present invention. As such, they should not be construed to limit the scope of the invention. Those in the art will well appreciate that many other embodiments also fall within the scope of the invention, as it is described hereinabove and in the claims.

### EXAMPLES

### Example 1

### HBV as a Target for RNAi-Based Therapeutics

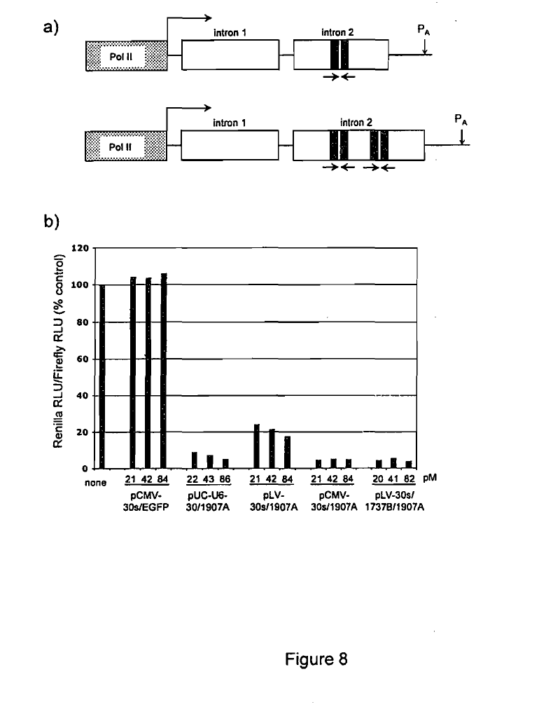

For therapeutic applications, interfering RNAs can be introduced into cells in several different ways. For example, synthetic short double-stranded RNAs (approximately 21 nucleotides) can be directly transfected into cells, where a single strand is incorporated into active RISC. Longer lasting silencing can be achieved by expressing interfering RNAs from viral or plasmid vectors. Typically, these expressed interfering RNAs (eiRNAs) are processed from short hairpin RNAs (shRNAs) transcribed by RNA polymerase III using U6, H1, or 7SK promoters. These promoters are strong, active in virtually all cell types, and well-adapted for transcription of short RNAs. See FIG. 8.

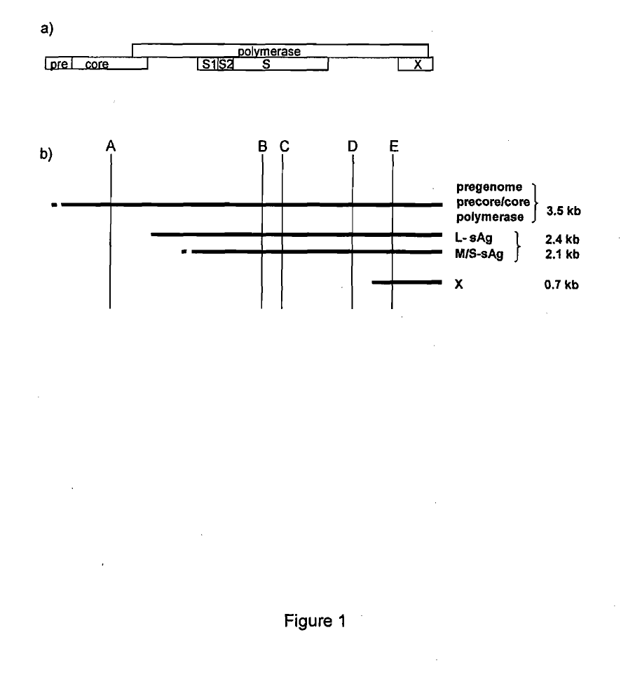

Previous work has identified regions in the HBV genome that are well-conserved among HBV serotypes and susceptible to silencing by expressed interfering RNAs (eiRNAs) [5]. These regions are found throughout the 3.2 kb genome and, because of the highly overlapping pattern of transcription, can include sequences that are simultaneously present on the 3.5 kb pregenomic RNA and one or more of the mRNAs that encode viral proteins, as depicted in FIG. 1. A plasmid vector has been generated for therapeutic applications, where four different expression cassettes, each using an RNA pol III promoter to drive transcription of an shRNA, have been combined in a single plasmid that provides potent silencing of HBV transcripts in cell culture assays and mouse models of HBV infection [5].

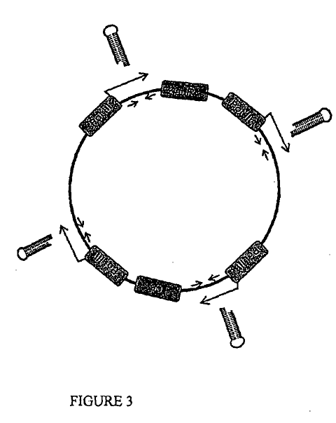

Vectors that express multiple shRNAs from a single plasmid were developed to maximize the efficacy of a clinical vector against a broad range of viral genotypes. This vector strategy is useful in minimizing the potential for selection of escape mutants, which is a serious limitation of some current HBV therapies based on nucleoside analog inhibitors of the viral polymerase. Such a vector was designed to express each shRNA from its own RNA polymerase III promoter (7SK or U6). See PCT/US2005/029976, which is herein incorporated by reference in its entirety. See FIG. 3.

Current HBV anti-viral agents reduce the burden of infectious virus, but are considerably less effective in decreasing viral antigenemia. High titers of subviral particles, consisting primarily of the viral surface antigen (HBsAg), are found in the serum of chronically infected patients. This is thought to contribute to viral persistence and to liver pathology. The ability of RNAi-based therapeutics to target the viral RNA pregenome as well as mRNAs encoding viral proteins offers a significant improvement over existing therapies. Reductions are seen in both RNA and protein products of viral replication, as well as in viral DNA replicative intermediates.

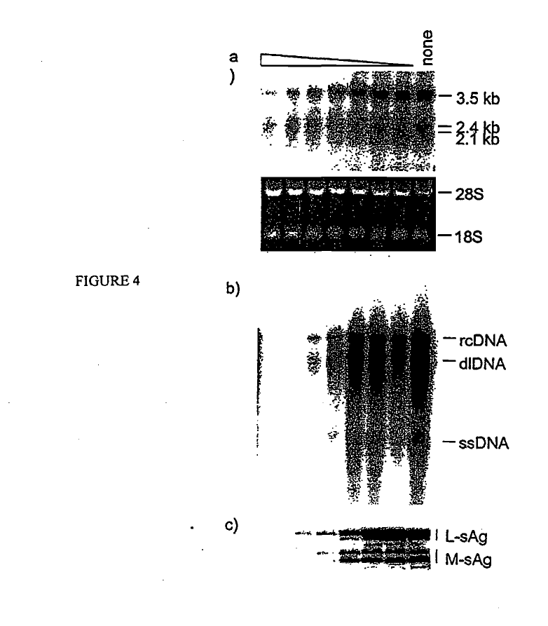

It has been observed that viral RNAs, DNA replicative intermediates, and surface antigen proteins are coordinately reduced in an infected cell (e.g. HepG2 cells transfected with pHBV2) when shRNAs were expressed from a vector containing the Pol III promoter (See FIG. 4).

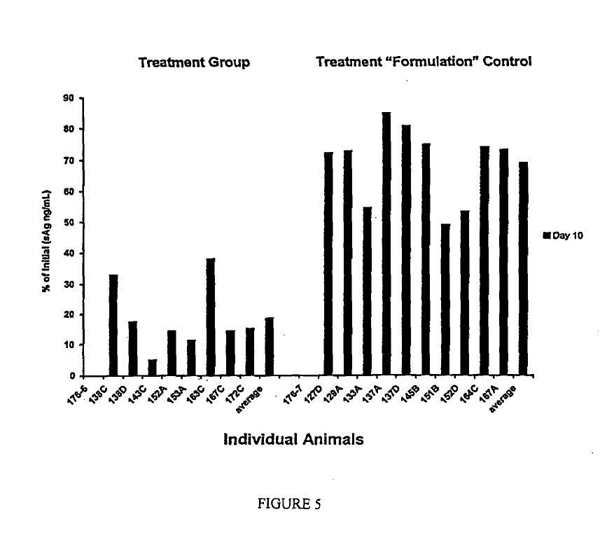

While shRNA expression plasmids can be highly effective suppressors of viral antigen production and infectious particle formation after transfection into cultured cells, in vivo delivery of these agents to hepatocytes remains a difficult challenge. The following experiments were designed to test the therapeutic effects of the RNAi-based silencing in an animal model. The shRNA expression plasmid was formulated with molecules that allowed transport to the liver and uptake by hepatocytes. Numerous formulations have been tested for delivery of shRNA expression plasmids to the liver in mouse models of HBV infection. See, e.g., U.S. Pat. No. 5,783,565; U.S. Pat. No. 7,202,227; U.S. Pat. No. 6,379,965; U.S. Pat. No. 6,127,170; U.S. Pat. No. 5,837,533, and US 2006/0084617, which are herein incorporated by reference in their entireties. Results obtained with a single dose using a cholesterol-spermine formulation are depicted in FIG. 5.

FIG. 5 demonstrates the reduction of serum HBsAg levels after intravenous delivery of dCS-formulated shRNA expression plasmid in a mouse model of HBV infection. HBsAg expression plasmid was hydrodynamically injected into SCID mice on day one of the experiment to establish expression of this antigen in the livers of the animals. Serum levels of HBsAg were measured on day 3. Subsequently, on day 5, formulated shRNA expression plasmid targeting HBsAg mRNAs was injected intravenously. Serum levels of HBsAg were again determined at various time points post-injection. Data obtained for individual animals is shown for day 10 of the experiment.

### Example 2

### Second Generation Vector Constructs

In the design of a second generation of vectors described here, we have used an miRNA format in order to capture the advantages of driving expression from a tissue specific RNA pol II promoter and processing multiple interfering RNAs from a single transcript that does not co-express any protein product.

Initially, we tested HBV-targeted interfering RNAs expressed from pol III promoters, but formatted as miRNAs instead of shRNAs. We based our constructs on the expression cassette found in Expression Arrest™ plasmids that use a U6 promoter to drive expression of interfering RNAs built into the context of human miR-30 [18]. To generate the pUC-U6-30/XX series of plasmids, we moved the expression cassette out of the pSM2 vector backbone and into the plasmid pUC19. The stem-loop region was then replaced with sequence targeting HBV RNAs in conserved regions near position 1737 or 1907 in the HBV genome.

The 1907 and 1737 regions of the HBV genome correspond approximately to D (1907) and E (1737) in FIG. 1. Therefore, D (1907) has the potential to target all major transcripts except the 0.7 kb RNA that encodes the X protein. E (1737) has the potential to target all transcripts. Note that both the viral pregenome (an RNA replicative intermediate) and the viral protein mRNAs would be targeted. Targets for both D and E lie in the polymerase coding region. The D target is also found in the 3′UTR of the mRNAs for L-, M-, and S-antigens. The E target is also in the 5′ UTR of the X mRNA. However, the location of the target with regard to the coding region for any given protein is probably irrelevant for silencing when the target and eiRNA are completely complementary.

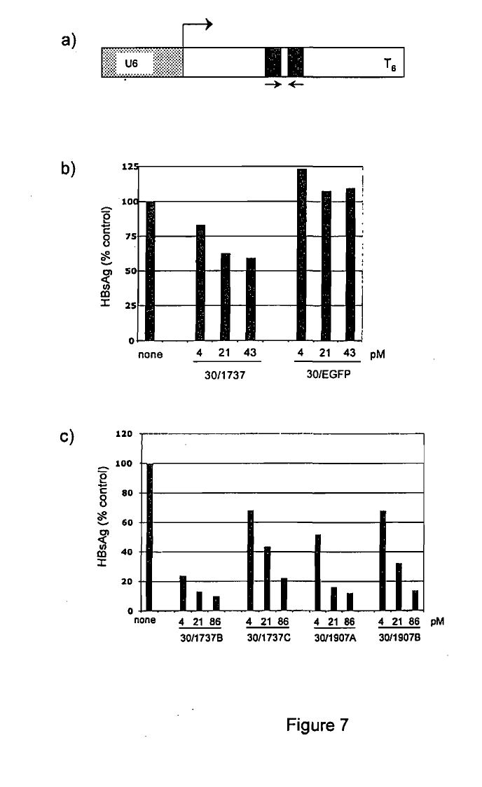

The efficacy of these plasmids was tested by assaying hepatitis S antigen (HBsAg) secretion from Huh7 cells after co-transfection with a constant amount of the infectious plasmid pHBV/2 and increasing amounts of silencing plasmid. A SEAP reporter plasmid, pM1-SEAP, was included to control for transfection efficiency. We found that direct transfer of sequence based on our shRNA plasmids [5] was not effective in the miRNA format. Results for the miRNA-formatted 1737 shRNA are shown in FIG. 7b, where secreted HBsAg is reduced only to ˜60% of control values, even at high doses of plasmid. To address this, conserved HBV sequence surrounding the original target region was searched using an si/miRNA design algorithm [19]. We found that silencing efficacy could be regained by redesigning the stem-loops to target a slightly shifted sequence in the HBV genome, but one that remains within the conserved regions. In the plasmids pUC-U6-30/1737B and -C, and pUC-U6-30/1907A and -B, the HBV target sequence was moved 3 to 10 bp relative to the original shRNA targets. FIG. 7c shows that these minor shifts can significantly alter the extent of silencing by the miR-formatted constructs, so that reductions of as much as 90% of HBsAg levels can be achieved even at intermediate doses of silencing plasmid. As expected, the silencing response is both dose and sequence dependent. The sequences of 1737B and -C, and 1907A and -B as compared to the original sequences, are as follows.

HBV genome target sequence (GenBank V01460). Targeted region is shown in bold, underlined print.

| | |
|---|---|
| a) | 1737 region (SEQ ID NO: 1) |
| | shRNA-1737: |
| | (bold region SEQ ID NO: 2) |
| | GGACGTCCTTTGTTTACGTCCCGTCGGCGCTGAATCC |
| | TGCGGACGACCCTTCT |
| | miR-1737B: |
| | (bold region SEQ ID NO: 3) |
| | GGACGTCCTTTGTTTACGTCCCGTCGGCGCTGAATCCTGCGGACGAC |
| | CCTTCT |
| | miR-1737C: |
| | (bold region SEQ ID NO: 4) |
| | GGACGTCCTTTGTTTACGTCCCGTCGGCGCTGAA |
| | TCCTGCGGACGACCCTTCT |
| b) | 1907 region (SEQ ID NO: 5) |
| | shRNA-1907: |
| | (bold region SEQ ID NO: 6) |
| | AACCTTTTCGGCTCCTCTGCCGATCCATACTGCGGAA |
| | CTCCTAGCCGCTTGTT |
| | miR-1907A: |
| | (bold region SEQ ID NO: 7) |
| | AACCTTTTCGGCTCCTCTGCCGATCCAT |
| | ACTGCGGAACTCCTAGCCGCTTGTT |
| | miR-1907B: |
| | (bold region SEQ ID NO: 8) |
| | AACCTTTTCGGCTCCTCTGCCGATCCATACTGCGGAACTCCTA |
| | GCCGCTTGTT |
| | miR-1907C: |
| | (bold region SEQ ID NO: 9) |
| | AACCTTTTCGGCTCCTCTGCCGATCCATAC |
| | TGCGGAACTCCTAGCCGCTTGTT |
| c) | 799 region (SEQ ID NO: 10) |
| | shRNA-799: |
| | (bold region SEQ ID NO: 11) |
| | CCCTAGAAGAAGAACTCCCTCGCCTCGCAGACGAAGGTCTCA |
| | ATCGCCGCGTCGCAGAAGA |
| | miR-799B: |
| | (bold region SEQ ID NO: 12) |
| | CCCTAGAAGAAGAACTCCCTCGCCTCGCAGACGAAGGTCTCAATC |
| | GCCGCGTCGCAGAAGA |
| d) | 2791 region (SEQ ID NO: 13) |
| | shRNA-2791 |
| | (bold region SEQ ID NO: 14) |
| | ACTTGTCCTGGTTATCGCTGGATGTGTCTGCGGCGTTTT |
| | ATCATCTTCCTCTTC |
| | miR-2791A |
| | (bold region SEQ ID NO: 15) |
| | ACTTGTCCTGGTTATCGCTGGATGTGTCTG |
| | CGGCGTTTTATCATCTTCCTCTTC |

Sequence of regions encoding HBV-targeted miRNAs (XbaI/SpeI restriction fragments):

1737B:
(SEQ ID NO: 16)
TCTAGAAGGCTAAAGAAGGTATATTGCTGTTGACAGTGAGCGAGCGCTG
AATCCTGCGGATGATTAGTGAAGCCACAGATGTAGTCGTCCGCAGGATT
CAGCGCCTGCCTACTGCCTCGGACTTCAAGGGGCTACTAGT
1737C:
(SEQ ID NO: 17)
TCTAGAAGGCTAAAGAAGGTATATTGCTGTTGACAGTGAGCGCTTACGT
CCCGTCGGCGCTGAATAGTGAAGCCACAGATGTATTCAGCGCCGACGGG
ACGTAAATGCCTACTGCCTCGGACTTCAAGGGGCTACTAGT
1907A:
(SEQ ID NO: 18)
TCTAGAAGGCTAAAGAAGGTATATTGCTGTTGACAGTGAGCGCTCGGCT
CCTCTGCCGATCCATTAGTGAAGCCACAGATGTAATGGATCGGCAGAGG
AGCCGAATGCCTACTGCCTCGGACTTCAAGGGGCTACTAGT
1907B:
(SEQ ID NO: 19)
TCTAGAAGGCTAAAGAAGGTATATTGCTGTTGACAGTGAGCGAATCCAT
ACTGCGGAACTCCTATAGTGAAGCCACAGATGTAGTATAGGAGTTCCGC
AGTATGGATCTGCCTACTGCCTCGGACTTCAAGGGGCTACTAGT
799B:
(SEQ ID NO: 20)
TCTAGAAGGCTAAAGAAGGTATATTGCTGTTGACAGTGAGCGATCGCAG
ACGAAGGTCTCAATCTAGTGAAGCCACAGATGTAGATTGAGACCTTCGT
CTGCGAGTGCCTACTGCCTCGGACTTCAAGGGGCTACTAGT
2791A:
(SEQ ID NO: 21)
TCTAGAAGGCTAAAGAAGGTATATTGCTGTTGACAGTGAGCGCGGTTAT
CGCTGGATGTGTCTGTAGTGAAGCCACAGATGTACAGACACATCCAGCG
ATAACCATGCCTACTGCCTCGGACTTCAAGGGGCTACTAGT

RNA Pol II Driven miR-eiRNAs

Several groups have reported successful silencing with plasmid or viral vectors that express miR-formatted interfering RNAs (miR-eiRNAs) from RNA pol II promoters. In general, however, the miR-eiRNAs were processed from a longer transcript that co-expresses a protein product. While this may have advantages in some research applications, it is undesirable for therapeutic eiRNAs where the co-expressed protein could be antigenic or toxic.

In our first attempts to introduce a pol II promoter into the pUC-U6-30/XX plasmids, we exchanged the U6 promoter for a liver specific promoter derived from the plasmid pLIVE. In addition we inserted a BGH 3′UTR downstream of the miR-30 3′ flanking sequence to prevent transcriptional readthrough into plasmid sequence. These constructs were inactive in silencing (data not shown). In order to correct this, we generated plasmids of the pLV-30s/XX series, where the eiRNA stem-loops are inserted into non-protein coding sequence, more closely mimicking natural miRNAs. For these plasmids, we moved the silencing stem-loop of pUC-U6-30/XX plasmids into the second intron of the pLIVE vector so that it would be processed from a transcript that consists primarily of two introns and contains no open reading frame for the production of a protein (see FIG. 8a). Only the stem-loop region and approximately 30 bp of miR-30 flanking sequence from the pUC-U6-30/XX plasmids were transferred (36 bp from the 5′ flanking side of miR-30 and 28 bp on the 3′ side). We also exchanged the liver specific promoter of the pLV-30s/XX plasmids for a CMV-IE promoter to create the pCMV-30s/XX series of plasmids.

Silencing activity of these pol II driven miR-eiRNAs was tested in Huh7 cells co-transfected with the silencing plasmid and a dual luciferase reporter plasmid. HBV target sequence was inserted into the 3′UTR of the Renilla luciferase gene cassette in the vector psiCHECK-2, while expression of firefly luciferase from the same plasmid serves as a transfection efficiency control. Results shown in FIG. 8b demonstrate that expression of 1907A HBV miR-eiRNA from the liver promoter (pLV-30s/1907A) is only slightly less effective in silencing than when it is expressed from a U6 promoter (pUC-U6-30/1907A). Use of the strong CMV pol II promoter leads to silencing that is equivalent to that seen with the U6 promoter. Importantly, the addition of a second stem-loop to the vector, targeting the 1737B region, increases the potency of silencing so that the pLV-30s/1737B/1907A plasmid is just as effective as the U6 or CMV driven 1907A plasmids. These data suggest that both miR-eiRNAs encoded in the bicistronic plasmid are functional and contribute to silencing of the target RNA.

Silencing Activity and Processing of Individual miR-eiRNAS Expressed from a Bicistronic Plasmid

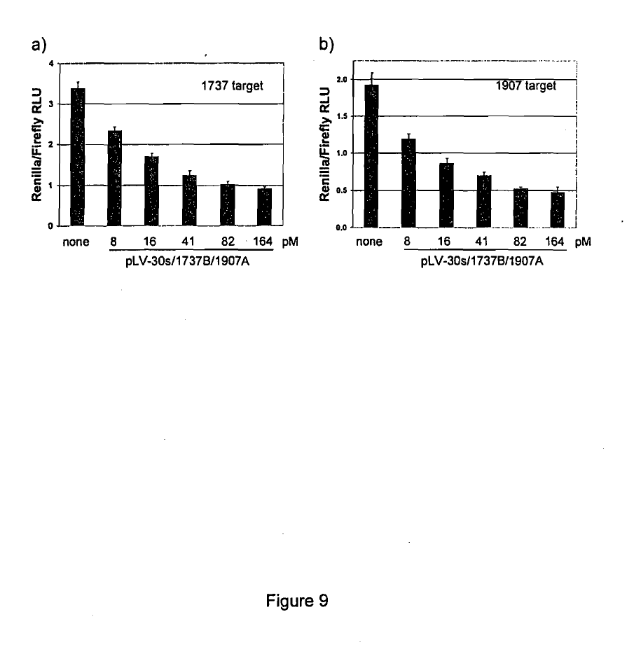

While the inclusion of a second miR-eiRNA stem-loop improved silencing of HBV targets, we sought direct evidence that each of the stem-loops in our bicistronic plasmid is active. Two dual luciferase reporter plasmids were constructed that contain HBV target sequence for either the 1737 or 1907 miR-eiRNAs, individually. As shown in FIG. 9, each of these reporter plasmids can be silenced by co-transfection with the bicistronic plasmid, pLV-30s/1737B/1907A, demonstrating that each of the miR-eiRNAs is functional.

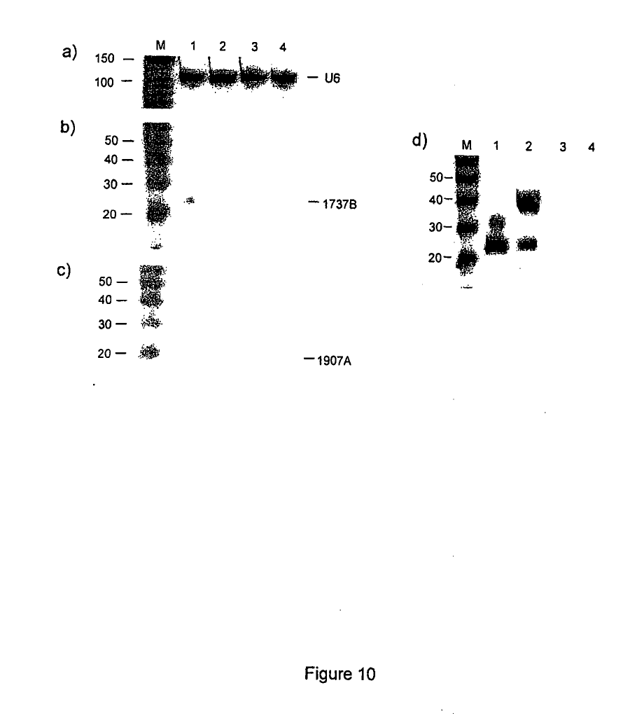

Additionally, we have examined the expression and processing of eiRNAs from the bicistronic plasmid in northern blots. Huh7 cells were transfected either with the monocistronic plasmids pLV-30s/1737B, pLV-30s/1907A, or pLV-30s/EGFP, or with the bicistronic plasmid pLV-30s/1737B/1097A. RNA isolated from these cells was analyzed by northern blotting, with sequential detection on the same blot using oligonucleotide probes for the anti-sense (guide) strand of each of the miRNAs. It is evident from the results shown in FIG. 10a-c that mature miRNAs are expressed and processed at similar levels from mono- and bi-cistronic plasmids.

The efficiency of processing of the miR-eiRNAs may help to account for their potency, despite their being expressed from pol II promoters that are generally less strong than pol III promoters. Results in FIG. 10d show that processing of the shRNA expressed from a pol III driven construct for 1737 eiRNA is incomplete (lane 2). In contrast, the pre-miRNA expressed from pUC-U6-30/1737A is processed almost completely into mature eiRNA (FIG. 10d, lane 1), as are the miR-eiRNAs expressed from pLV-30s/1737B and pLV-30s/1907A, shown in FIG. 10a-c.

### Example 3

### Tissue-Specific Silencing from the RNA Pol II Promoter

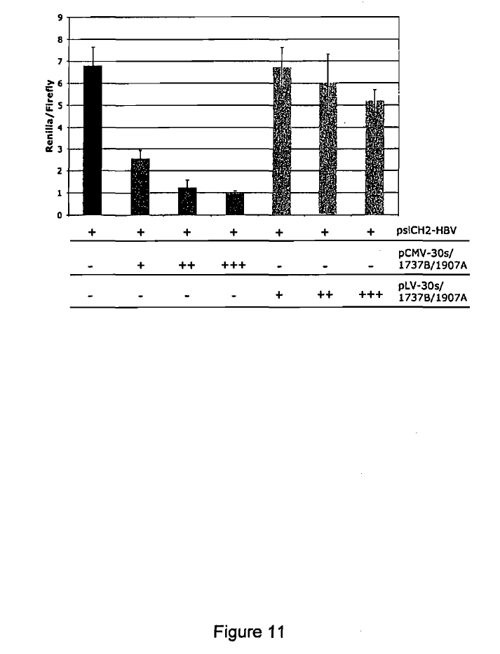

In using a pol II promoter, we expect to be able to select promoters that will enhance the relative expression of miR-eiRNAs in targeted tissues. For HBV therapeutic eiRNAs it will be advantageous to maximize expression in hepatocytes and minimize expression in other tissues, thereby reducing concerns about potentially deleterious effects in non-targeted tissues. The liver specific promoter from pLIVE, used in our pLV-30s/XX plasmids, combines a mouse alpha-fetoprotein enhancer and minimal albumin promoter. To test the tissue specificity of silencing with this promoter, we compared silencing in HeLa cells transfected with plasmids carrying the liver-specific promoter (pLV-30s/1737B/1907A) to that with the broadly active CMV promoter (pCMV-30s/1737B/1907A). FIG. 11 shows that the HBV miR-eiRNAs are capable of strong silencing of an HBV reporter plasmid in these cells, when expressed from a CMV promoter. However, very little silencing was achieved with plasmids using the liver-specific promoter in HeLa cells, most likely due to low levels of expression of the miR-eiRNAs.

### Example 4

### Expression of miR-eiRNAs does not Induce an Interferon Response

The expression of interfering RNAs in cells, whether formatted as sh- or mi-RNAs, leads to the production of highly structured RNAs that have the potential to induce an interferon response. To test whether the miR-eiRNAs produced from our bicistronic plasmid trigger an interferon response, we transfected cells and assayed several mRNAs that serve a markers for this response. In these experiments, we used HeLa cells since they are capable of a more robust interferon response than Huh7 cells. The plasmid pCMV-30s/1737B/1907A was transfected into cells in an amount higher than required for >85% reduction in HBV reporter activity (see FIG. 11). Quantitative RT-PCR was used to determine levels of mRNA encoding p56 (IFIT-1), IFN-β, and MX-1 in transfected cells relative to levels in untreated HeLa cells.

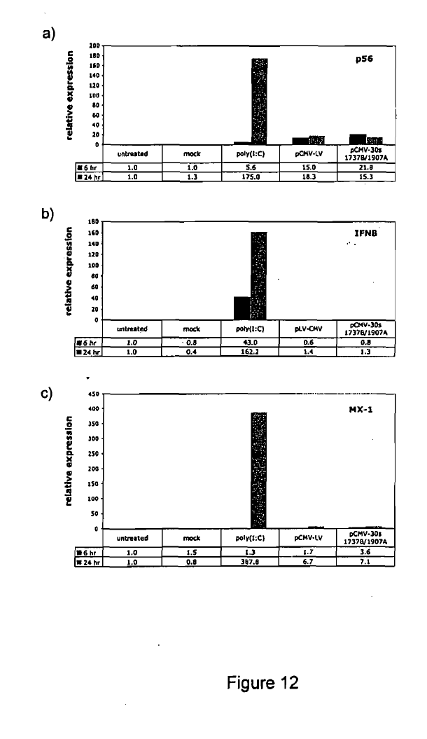

As shown in FIG. 12, very little induction of any of these markers was observed at either 6 hr or 24 hr post-transfection, as compared to the response induced by transfection with poly(I:C), used as a positive control. In fact, even the minimal induction of p56, the most sensitive of the markers tested, appears to have been caused by introduction of the vector itself, not by expression of the miR-eiRNAs. After transfection with the empty vector, pCMV-LV, that produces no miR-eiRNA, the low level of p56 induction is comparable to that seen when pCMV-30s/1737B/1907A is transfected (see FIG. 12a). We conclude that, at levels above those necessary for efficacy in silencing HBV targets, there is no significant induction of an interferon response by the expression of the HBV targeted miR-eiRNAs.

### Example 5

### Expression of miR-eiRNAs in NOD-SCID Mice

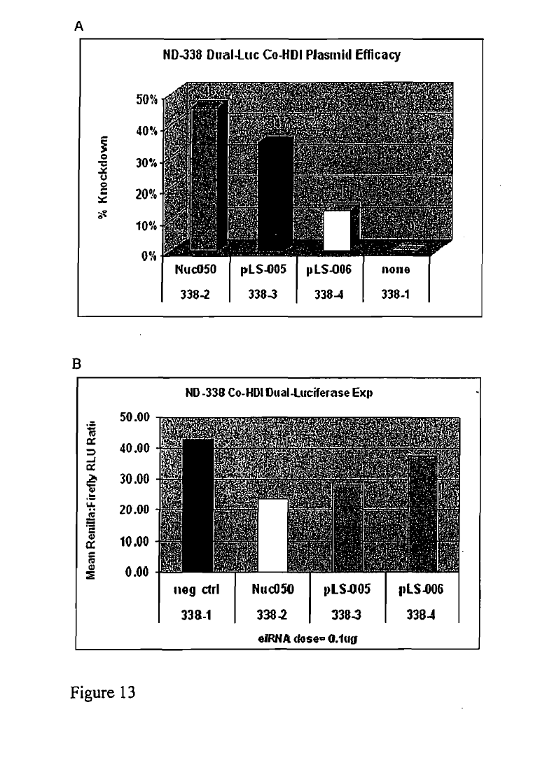

The indicated silencing plasmids (FIG. 13) were coinjected with a reporter target plasmid (based on psiCHECK2) that contains all four of the regions targeted by Nuc050, the multi-genic sh-eiRNA silencing plasmid shown in FIG. 3. Only two of these regions are targeted by the miR-eiRNA constructs LS-005 (pLV-30s/1737B/1907A) and LS-006 (pCMV-30s/1737B/1907A). The reporter target plasmid together with 0.1 μg of silencing plasmid was introduced into NOD-SCID mice by hydrodynamic injection into the tail vein. Groups of 10 mice, or 8 mice for LS-005, were injected for each plasmid and livers were collected 5 days post-injection for assay of Renilla and firefly luciferase. Silencing activity was measured as a reduction in Renilla luciferase activity relative to firefly luciferase activity.

As can be seen in graphs A and B of FIG. 13, the pLV-30s/1737B/1907A inhibited target gene expression more effectively than the same construct with the CMV promoter at the time point measured. Results in table format are presented below. The Nuc050 vector expressing four separate shRNAs was more effective than pLV-30s/1737B/1907A, which is not surprising given that the Nuc050 vector expresses two more targeting constructs as compared to pLV-30s/1737B/1907A. Further miRNA units incorporated into pLV-30s/1737B/1907A should enhance the efficacy of the miR-eiRNA as compared to Nuc050.

TABLE 1
Results of miR-eiRNA silencing in NOD-SCID mice

| # | plasmid | Mean R:F | % Expression | % Knockdown |
|---|---|---|---|---|
| 338-2 | Nuc050 | 23.4 | 54% | 46% |
| 338-3 | pLS-005 | 28.3 | 66% | 34% |
| 338-4 | pLS-006 | 37.5 | 87% | 13% |
| 338-1 | none | 43.1 | 100% | 0% |

### Example 6

### Multicistronic Plasmids Expressing miR-eiRNAs Show More Potent Silencing than Bicistronic Plasmids Expressing miR-eiRNAs

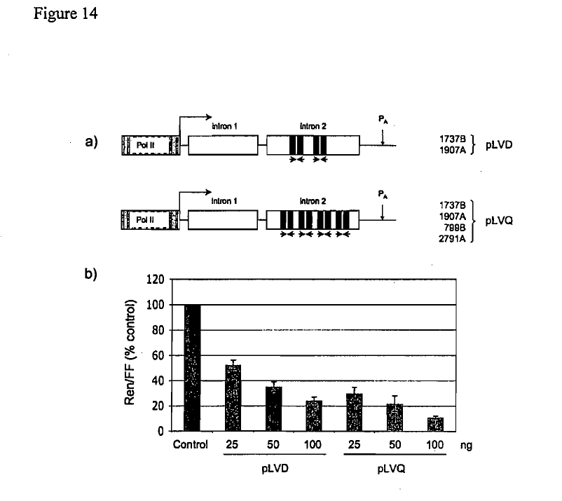

To examine whether inclusion of additional miR-eiRNA stem-loop structures would enhance the suppression of target gene expression, we constructed a plasmid expressing four different miR-eiRNAs under the control of a liver-specific promoter so that four distinct regions of the HBV genome were targeted. In addition to the pLVD construct expressing 1737B and 1907A HBV miR-eiRNAs, we added two additional miR-eiRNAs targeting the 799B (SEQ ID NO: 12) and 2791A (SEQ ID NO: 15) regions of the HBV genome (pLVQ construct, see FIG. 14a). The silencing activity of the pLVD and pLVQ plasmids was tested in Huh7 cells co-transfected with a constant amount of the dual luciferase reporter, psiCH-HBV, and increasing amounts of pLVD or pLVQ. Results are shown in FIG. 14b and are expressed as the ratio of Renilla to firefly luciferase activity, normalized to results from cells with no added silencing plasmid (“control”). These results show that multiple microRNA-formatted interfering RNAs can be expressed from the same promoter to produce an enhanced silencing of target genes.

miR-eiRNAs Expressed from Exons are as Effective as miR-eiRNAs Expressed from Introns

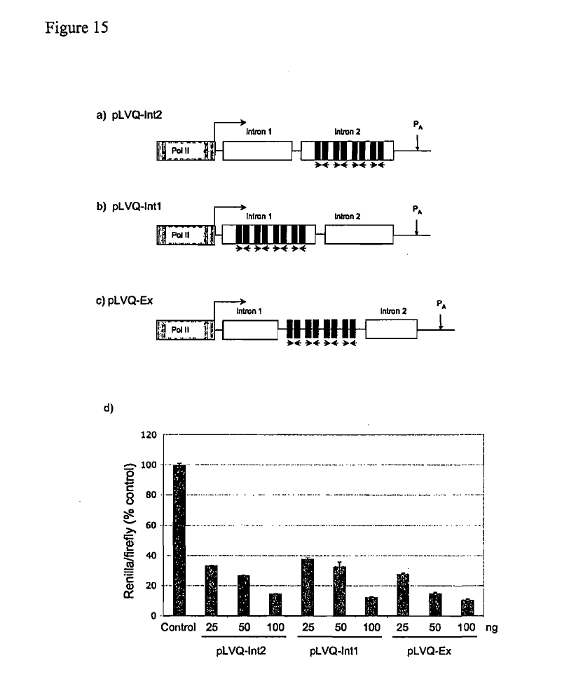

Four miR-eiRNAs targeting the 1737B, 1907A, 799B, and 2791A regions of the HBV genome were placed either into the second intron (pLVQ-Int2, FIG. 15a), into the first intron (pLVQ-Int1, FIG. 15b), or into the exon region located between the two introns (pLVQ-Ex, FIG. 15c) in expression constructs under the control of a liver-specific promoter. The construct did not contain an open reading frame for the production of a protein. Silencing activity of the different plasmids was tested by co-transfecting Huh7 cells with a constant amount of the dual luciferase reporter, psiCH-HBV, and increasing amounts of each of the indicated silencing plasmids. Results from this set of experiments is shown in FIG. 15d. Silencing activity is expressed as the ratio of Renilla to firefly luciferase activity, normalized to results from cells with no added silencing plasmid (“control”). The miR-formatted interfering RNAs expressed from the exonic region were as effective in silencing target genes as those expressed from either of the introns.

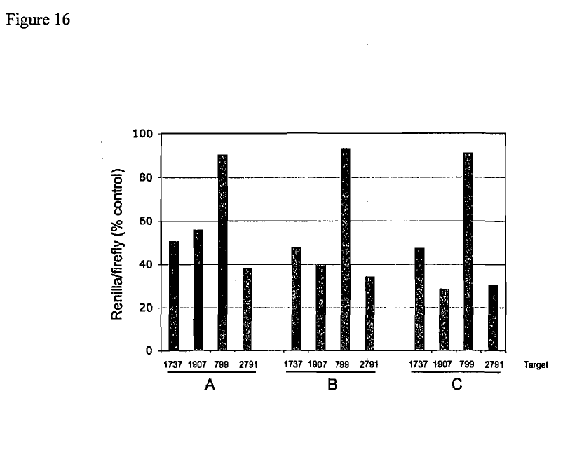

To ensure that no loss of function occurred when individual miR-eiRNAs were expressed from a multi-cistronic plasmid as compared to a monocistronic plasmid, a dual luciferase reporter construct containing a HBV target sequence for 1737, 1907, 799, or 2791 (as indicated along the X axis in FIG. 16) was co-transfected in Huh7 cells with either the corresponding monocistronic silencing plasmid expressing the miR-eiRNA for the targeted region (section A), the multi-cistronic pLVQ-Int2 (section B), or the multi-cistronic pLVQ-Ex (section C). The results indicate that miR-eiRNA expressed from either the exonic or intronic regions of multi-cistronic plasmids were as effective at suppressing target gene expression as miR-eiRNA expressed from monocistronic plasmids. In some cases (e.g. 1907 and 2791), miR-eiRNA expressed from the multi-cistronic plasmid showed enhanced target gene silencing compared to expression from the monocistronic plasmid.

### REFERENCES

1. Lee, W. M. (1997). Hepatitis B virus infection. New Engl J Med 337: 1733-1745.
2. WHO (2000). Hepatitis B, Fact Sheet No. 204. http://www.who.int/mediacentre/factsheets/fs204/en/.
3. Lok, A. S. F., and McMahon, B. J. (2007). Chronic hepatitis B. Hepatology 45: 507-539.
4. Zoulim, F. (2006). Antiviral therapy of chronic hepatitis B. Antiviral Research 71: 206-215.
5. Romano, P. R., McCallus, D. E., and Pachuk, C. J. (2006). RNA interference-mediated prevention and therapy for hepatocellular carcinoma. Oncogene 25: 3857-3865.
6. Chisari, F. V., and Ferrari, C. (1995). Hepatitis B virus immunopathogenesis. Annu Rev Immunol 13: 29-60.
7. Radhakrishnan, S. K., Layden, T. J., and Gartel, A. L. (2004). RNA interference as a new strategy against viral hepatitis. Virology 323: 173-181.
8. Hamasaki, K., Nakao, K., Matsumoto, K., Ichikawa, T., Ishikawa, H., and Eguchi, K. (2003). Short interfering RNA-directed inhibition of hepatitis B virus replication. FEBS Letters 543: 51-54.
9. Liu, J., et al. (2004). Effect of vector-expressed siRNA on HBV replication in hepatoblastoma cells. World J Gastroenterol 10: 1898-1901.
10. Moore, M. D., McGarvey, M. J., Russell, R. A., Cullen, B. R., and McClure, M. O. (2005). Stable inhibition of hepatitis B virus proteins by small interfering RNA expressed from viral vectors. J Gene Med 7: 918-925.
11. Shlomai, A., and Shaul, Y. (2003). Inhibition of hepatitis B virus expression and replication by RNA interference. Hepatology 37: 764-770.
12. Zhang, X.-N., Xiong, W., Wang, J.-D., Hu, Y.-W., Xiang, L., and Yuan, Z.-H. (2004). siRNA-mediated inhibition of HBV replication and expression. World J Gastroenterol 10: 2967-2971.
13. Giladi, H., Ketzinel-Gilad, M., Rivkin, L., Felig, Y., Nussbaum, O., and Galun, E. (2003). Small interfering RNA inhibiits hepatitis B virus replication in mice. Molecular Therapy 8: 769-776.
14. McCaffrey, A. P., et al. (2003). Inhibition of hepatitis B virus in mice by RNA interference. Nature Biotechnology 21: 639-644.
15. Morrissey, D. V., et al. (2005). Activity of stabilized short interfering RNA in a mouse model of hepatitis B virus replication. Hepatology 41: 1349-1356.
16. Uprichard, S. L., Boyd, B., Althage, A., and Chisari, F. V. (2005). Clearance of hepatitis B virus from the liver of transgenic mice by short hairpin RNAs. Proc. Natl. Acad. Sci. USA 102: 773-778.
17. Ying, R. S., et al. (2007). Hepatitis B virus is inhibited by RNA interference in cell culture and in mice. Antiviral Research 73: 24-30.
18. Silva, J. M., et al. (2005). Second-generation shRNA libraries covering the mouse and human genomes. Nat Genet 37: 1281-1288.
19. Hannon, G. J. (2006). http://katahdin.cshl.org:9331/homepage/siRNA/RNAi.cgi?type=siRNA.
20. Dykxhoorn, D. M., and Lieberman, J. (2005). The silent revolution: RNA interference as basic biology, research tool, and therapeutic. Ann Rev Med 56: 401-423.
21. Caplen, N. J., Parrish, S., Imani, F., Fire, A., and Morgan, R. A. (2001). Specific inhibition of gene expression by small double-stranded RNAs in invertebrate and vertebrate systems. Proc Natl Acad Sci USA 98: 9742-9747.
22. Elbashir, S. M., Harborth, J., Lendeckel, W., Yalcin, A., Weber, K., and Tuschl, T. (2001). Duplexes of 21-nucleotide RNAs mediate RNA interference in cultured mammalian cells. Nature 411: 494-498.
23. Jackson, A. L., et al. (2006). Position-specific chemical modification of siRNAs reduces “off-target” transcript silencing. RNA 12: 1197-1205.
24. Morrissey, D. V., et al. (2005). Potent and persistent in vivo anti-HBV activity of chemically modified siRNAs. Nature Biotechnol 23: 1002-1007.
25. Zimmermann, T. S., et al. (2006). RNAi-mediated gene silencing in non-human primates. Nature 441: 111-114.
26. Brummelkamp, T. R., Bernards, R., and Agami, R. (2002). A system for stable expression of short interfering RNAs in mammalian cells. Science 296: 550-553.
27. Paddison, P. J., Caudy, A. A., Bernstein, E., Hannon, G. J., and Conklin, D. S. (2002). Short hairpin RNAs (shRNAs) induce sequence-specific silencing in mammalian cells. Genes & Devel 16: 948-958.
28. Paul, C. P., Good, P. D., Winer, I., and Engelke, D. R. (2002). Effective expression of small interfering RNA in human cells. Nature Biotechnology 29: 505-508.
29. Yu, J. Y., DeRuiter, S. L., and Turner, D. L. (2002). RNA interference by expression of short-interfering RNAs and hairpin RNAs in mammalian cells. Proc Natl Acad Sci USA 99: 6047-6052.
30. Boden, D., Pusch, O., Silbermann, R., Lee, F., Tucker, L., and Ramratnam, B. (2004). Enhanced gene silencing of HIV-1 specific siRNA using microRNA designed hairpins. Nucl. Acids Res. 32: 1154-1158.
31. McManus, M. T., Petersen, C. P., Haines. B. B., Chen, J., and Sharp, P. (2002). Gene silencing using micro-RNA designed hairpins. RNA 8: 842-850.
32. Zeng, Y., Wagner, E. J., and Cullen, B. R. (2002). Both natural and designed micro RNAs can inhibit the expression of cognate mRNAs when expressed in human cells. Molecular Cell 9: 1327-1333.
33. Altuvia, Y., et al. (2005). Clustering and conservation patterns of human microRNAs. Nucleic Acids Research 33: 2697-2706.
34. Lagos-Quintana, M., Rauhut, R., Lendeckel, W., and Tuschl, T. (2001). Identification of novel genes coding for small expressed RNAs. Science 294: 853-858.
35. Lee, Y., Jeon, K., Lee, J.-T., Kim, S., and Kim, V. N. (2002). MicroRNA maturation: stepwise processing and subcellular localization. EMBO J 21: 4663-4670.
36. Cai, X., Hagedorn, C. H., and Cullen, B. R. (2004). Human microRNAs are processed from capped, polyadenylated transcripts that can also function as mRNAs. RNA 10: 1957-1966.
37. Lee, Y., et al. (2004). MicroRNA genes are transcribed by RNA polymerase II. EMBO J 23: 4051-4060.
38. Han, J., et al. (2006). Molecular basis for the recognition of primary microRNAs by the Drosha-DGCR8 complex. Cell 125: 887-901.
39. Zeng, Y., Yi, R., and Cullen, B. R. (2005). Recognition and cleavage of primary microRNA precursors by the nuclear processing enzyme Drosha. EMBO J 24: 138-148.
40. Dickins, R. A., et al. (2005). Probing tumor phenotypes using stable and regulated synthetic microRNA precursors. Nat Genet 37: 1163-1165.
41. Stegmeier, F., Hu, G., Rickles, R. J., Harmon, G. J., and Elledge, S. J. (2005). A lentiviral microRNA-based system for single-copy polymerase II-regulated RNA interference in mammalian cells. Proc. Natl. Acad. Sci. USA 102: 13212-13217.
42. Chung, K.-H., et al. (2006). Polycistronic RNA polymerase II expression vectors for RNA interference based on BIC/miR-155. Nucleic Acids Research 34: e53.
43. Sun, D., Melegari, M., Sridhar, S., Rogler, C. E., and Zhu, L. (2006). Multi-miRNA hairpin method that improves gene knockdown efficiency and provides linked multi-gene knockdown. Biotechniques 41: 59-63.
44. Xia, X.-G., Zhou, H., and Xu, Z. (2006). Multiple shRNAs expressed by an inducible pol II promoter can knock down the expression of multiple target genes. Biotechniques 41: 64-68.
45. ter Brake, O., Konstantinova, P., Ceylan, M., and Berkhout, B. (2006). Silencing of HIV-1 with RNA interference: a multiple shRNA approach. Molecular Therapy 14: 883-892.
46. Schubert, S., Grunert, H. P., Zeichhardt, H., Werk, D., Erdmann, V. A., and Kurreck, J. (2005). Maintaining inhibition: shRNA double expression vectors against coxsackieviral RNAs. J Mol Biol 346: 457-465.
47. Sioud, M. (2006). RNA interference below the immune radar. Nature Biotechnology 24: 521-522.
48. Cullen and Zeng, US patent application US 2004/0053411 (published Mar. 18, 2004).
49. Turner and Yu, US patent application US 2004/0053876 (published Mar. 18, 2004).
50. Zhou, H., ia, X. G. and Xu, Z. (2005) An RNA polymerase II construct synthesizes short-hairpin RNA with a quantitative indicator and mediates highly efficient RNAi. Nuc Acids Res 33, e62.
51. Denli A M, Tops B B, Plasterk B B, Ketting R F, Hannon G J. (2004). Nature 432(7014):231-5.
52. Han J, Lee Y, Yeom K H, Nam J W, Heo I, Rhee J K, Sohn S Y, Cho Y, Zhang B T, Kim V N. (2006). Molecular basis for the recognition of primary microRNAs by the Drosha-DGCR8 complex. Cell 125(5):887-901.
53. Rodriguez, A., Griffiths-Jones, S., Ashurst, J. L., and Bradley, A. (2006). Identification of mammalian microRNA host genes and transcription units. Genome Research 14: 1902-1910.

All publications, patents and patent applications discussed and cited herein are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1-42. (canceled)

43. A nucleic acid vector comprising a nucleic acid encoding at least two microRNA (miRNA)-formatted interfering RNAs specific for at least one target gene, wherein said nucleic acid encoding said at least two miRNA-formatted interfering RNAs is operably linked to a pol III promoter.

44. The nucleic acid vector of claim 43, wherein said pol III promoter is the U6 promoter.

45. A method of inhibiting or decreasing expression of at least one target gene in a cell in vitro comprising transfecting said cell with the nucleic acid vector of claim 43 such that said nucleic acid encoding at least two miRNA-formatted interfering RNAs is expressed and expression of said at least one target gene is inhibited or decreased.

46. A method of inhibiting or decreasing expression of at least one target gene in a mammalian cell in vivo comprising delivering to said cell the nucleic acid vector of claim 43 such that said nucleic acid encoding at least two miRNA-formatted interfering RNAs is expressed and expression of said at least one target gene is inhibited or decreased.

47. A method of treating a patient infected with HBV or HCV comprising delivering to the liver of said patient the nucleic acid vector of claim 66 such that said nucleic acid encoding said at least two miRNA-formatted interfering RNAs is expressed and expression of at least one HBV or HCV gene is inhibited or decreased.

48. A nucleic acid vector comprising a nucleic acid encoding at least two microRNA (miRNA)-formatted interfering RNAs specific for at least one target gene, wherein said nucleic acid encoding said at least two miRNA-formatted interfering RNAs is operably linked to a single tissue-specific pol II promoter, and wherein said nucleic acid vector decreases expression of said at least one target gene in a mammalian cell at least as effectively or more effectively than a vector expressing either of said miRNA-formatted interfering RNAs alone.

49. The nucleic acid vector of claim 48, wherein said nucleic acid encoding said at least two miRNA-formatted interfering RNAs is not operably linked to a separate protein coding sequence.

50. The nucleic acid vector of claim 48, wherein said nucleic acid encoding said at least two miRNA-formatted interfering RNAs is located in a nucleic acid encoding an intron or in a nucleic acid encoding an untranslated region of an mRNA, or in a non-coding RNA.

51. The nucleic acid vector of claim 48, wherein said nucleic acid encoding said at least two miRNA-formatted interfering RNAs is located in a nucleic acid encoding an exon.

52. The nucleic acid vector of claim 48, wherein said at least two miRNA-formatted interfering RNAs each contain a region capable of forming a stem-loop structure, wherein the stem is flanked by 5′ and 3′ arm regions derived from a naturally occurring miRNA.

53. The nucleic acid vector of claim 48, wherein said at least two (miRNA)-formatted interfering RNAs each contain a region capable of forming a stem-loop structure, wherein the stem is flanked by 5′ and 3′ arm regions having a secondary structure modeled after a naturally occurring miRNA.

54. The nucleic acid vector of claim 52, wherein said 3′ arm regions of said at least two miRNA-formatted interfering RNAs contain no more than about 20 to about 45 consecutive nucleotides.

55. The nucleic acid vector of claim 52, wherein said 5′ arm regions of said at least two miRNA-formatted interfering RNAs contain no more than about 20 to about 45 consecutive nucleotides.

56. The nucleic acid vector of claim 52, wherein the loop portions of each of said at least two miRNA-formatted interfering RNAs are derived from a naturally occurring miRNA.

57. The nucleic acid vector of claim 52, wherein said regions capable of forming the stem-loop structures each contain a stem portion of about 20 to about 25 base pairs, wherein the two strands of each stem are at least substantially complementary to each other.

58. The nucleic acid vector of claim 57, wherein the stern strands of each of said at least two miRNA-formatted interfering RNAs are completely complementary to one another.

59. The nucleic acid vector of claim 58, wherein processing of said at least two microRNA-formatted interfering RNAs results in degradation of the mRNA transcripts encoded by the one or more target gene(s) or translational arrest of the target mRNA transcript(s).

60. The nucleic acid vector of claim 52, wherein said naturally occurring miRNA naturally contains only one sequence capable of forming a stem-loop structure.

61. The nucleic acid vector of claim 60, wherein said miRNA is miR-30.

62. The nucleic acid vector of claim 61, wherein each miRNA-formatted interfering RNA contains a region capable of forming a stem-loop structure, wherein the stem is flanked by 5′ and 3′ arm regions derived from a naturally occurring miRNA that are each no more than about 25 to about 40 consecutive nucleotides, and wherein said nucleic acid regions encoding each miRNA-formatted interfering RNA are operably linked with no more than about 6 to 10 nucleotides.

63. The nucleic acid vector of claim 48, wherein said at least two miRNA-formatted interfering RNAs are specific for at least one pathogen target gene.

64. The nucleic acid vector of claim 48, wherein said at least two miRNA-formatted interfering RNAs are specific for at least two pathogen target genes.

65. The nucleic acid vector of claim 48, wherein said at least two miRNA-formatted interfering RNAs are specific for target genes of two or more pathogens.

66. The nucleic acid vector of claim 48, wherein said tissue-specific promoter is a liver-specific promoter.

67. The nucleic acid vector of claim 66, wherein said at least two miRNA-formatted interfering RNAs are specific for at least one pathogen target gene.

68. The nucleic acid vector of claim 67, wherein said pathogen is selected from the group consisting of HBV and HCV.

69. The nucleic acid vector of claim 63, wherein said at least one pathogen gene is an influenza virus gene.

70. The nucleic acid vector of claim 63, wherein said at least one pathogen gene is an HIV gene.

71. The nucleic acid vector of claim 48, wherein said at least two miRNA-formatted interfering RNAs are specific for at least two target genes.

72. The nucleic acid vector of claim 48 further comprising a third miRNA-formatted interfering RNA.

73. The nucleic acid vector of claim 72 further comprising a fourth miRNA-formatted interfering RNA.

74. The nucleic acid vector of claim 73, wherein said nucleic acid encoding the four miRNA-formatted interfering RNAs is located in a nucleic acid encoding an intron, an exon, or in a nucleic acid encoding an untranslated region of an mRNA, or in a non-coding RNA.

75. A method of inhibiting or decreasing expression of at least one target gene in a cell in vitro comprising transfecting said cell with the nucleic acid vector of claim 48 such that said nucleic acid encoding at least two miRNA-formatted interfering RNAs is expressed and expression of said at least one target gene is inhibited or decreased.

76. A method of inhibiting or decreasing expression of at least one target gene in a mammalian cell in vivo comprising delivering to said cell the nucleic acid vector of claim 48 such that said nucleic acid encoding at least two miRNA-formatted interfering RNAs is expressed and expression of said at least one target gene is inhibited or decreased.

77. A method of treating a patient infected with HBV or HCV comprising delivering to the liver of said patient the nucleic acid vector of claim 68 such that said nucleic acid encoding said at least two miRNA-formatted interfering RNA is expressed and expression of at least one HBV or HCV gene is inhibited or decreased.

78. A method of treating a patient infected with HBV or HCV comprising delivering to the liver of said patient the nucleic acid vector of claim 71 such that said nucleic acid encoding said at least two miRNA-formatted interfering RNAs is expressed and expression of at least one HBV or HCV gene is inhibited or decreased.

79. A nucleic acid vector for expressing one or more miRNA-formatted interfering RNAs comprising a tissue-specific pol II promoter operably linked to first and second intron sequences and a polyadenylation signal, wherein said second intron comprises 5′ and 3′ miRNA-formatted arm regions of no more than about 20 to about 45 consecutive nucleotides each flanking one or more cloning sites.

80. The nucleic acid vector of claim 79, wherein said 5′ and 3′ arm regions are derived from a naturally occurring miRNA.

81. The nucleic acid vector of claim 79, wherein said 5′ and 3′ arm regions have a secondary structure modeled after a naturally occurring miRNA.

82. The nucleic acid vector of claim 80, wherein said naturally occurring miRNA is miR-30.

83. The nucleic acid vector of claim 81, wherein said naturally occurring miRNA is miR-30.

84. The nucleic acid vector of claim 82, wherein said 5′ arm region contains no more than 35 to 40 nucleotides.

85. The nucleic acid vector of claim 82, wherein said 3′ arm region contains no more than 25 to 30 nucleotides.

86. The nucleic acid vector of claim 79, further comprising one or more cloning restriction sites 5′ and 3′ of said miRNA-formatted arm regions.

87. The nucleic acid vector of claim 86, wherein said cloning sites are XbaI and SpeI.

88. A nucleic acid vector for expressing one or more miRNA-formatted interfering RNAs comprising a tissue-specific pol II promoter operably linked to first and second intron sequences and a polyadenylation signal, wherein said first intron comprises 5′ and 3′ miRNA-formatted arm regions of no more than about 20 to about 45 consecutive nucleotides each flanking one or more cloning sites.

89. A nucleic acid vector for expressing one or more miRNA-formatted interfering RNAs comprising a tissue-specific pol II promoter operably linked to first and second intron sequences separated by an exon sequence and a polyadenylation signal, wherein said exon sequence comprises 5′ and 3′ miRNA-formatted arm regions of no more than about 20 to about 45 consecutive nucleotides each flanking one or more cloning sites.
